(19) United States
(12) Patent Application Publication
(10) Pub. No.: US 2023/0233513 A1
(43) Pub. Date: Jul. 27, 2023

(54) TAXIFOLIN FORMULATION COMPRISING THIAMINE

(71) Applicant: Evanium Healthcare GmbH, Regensburg (DE)

(72) Inventors: David OFNER, Regensburg (DE); Felix Carlo WERKMANN, München (DE); Felix ROLKA, Aachen (DE)

(73) Assignee: Evanium Healthcare GmbH, Regensburg (DE)

(21) Appl. No.: 17/919,010

(22) PCT Filed: Apr. 15, 2021
(86) PCT No.: PCT/EP2021/059761

§ 371 Date: Oct. 14, 2022

(30) Foreign Application Priority Data
Apr. 16, 2020 (EP) 20169873.5

(51) Int. Cl. A61K 31/352; A61K 9/00; A61K 47/61; A61K 31/51; A61K 36/15; A61K 47/32; A61K 9/20

(52) U.S. Cl. CPC A61K 31/352; A61K 9/0053; A61K 47/61; A61K 31/51; A61K 36/15; A61K 47/32; A61K 9/2054; A61K 9/2031

(57) ABSTRACT

The present invention relates to formulations of taxifolin with thiamine as a dosage form for oral administration, in particular as dietary supplements or foods for special medical purposes/FSMPs.

The present invention relates to formulations of taxifolin with thiamine as a dosage form for oral administration, in particular as a dietary supplement.

Alcohol intoxications and the damage associated therewith, as well as the side effects on the day after alcohol consumption, are a widespread problem that is difficult to control. This is due in part to the complex mechanism of action underlying drinking alcohol (ethanol). Unlike benzodiazepines, for example, being a very small molecule, alcohol is capable of exerting its effect at various binding sites of the responsible receptor. In particular, the GABA_{A} receptor is responsible for most of the alcohol effects. This ionotropic receptor consists of five subunits (two α, two β, one γ/δ/ε/θ/γ), wherein tonic receptors, which consist of a δ subunit in combination with two α4 or α6 and two β3 subunits, respectively, react particularly sensitive to ethanol.

Certain flavonoids, based on the structure of the flavonoid taxifolin have a positive effect on alcohol consumption, in particular with regard to neurological damage as well as alcohol-related consequential conditions such as hangover symptoms. This is due to an interaction with ethanol-sensitive GABA_{A} receptors, whereby it was found for the first time that these flavonoids specifically act as negative modulators. For this purpose, the flavonoids are used in the form of a complex with β-cyclodextrin or as a solid dispersion in basic polymethacrylate, respectively, since surprisingly, only in this formulation a significant effect could be found.

Now, surprisingly, it has been found that the nutritional application in oral form can be significantly improved by the addition of thiamine.

Thiamine is also known as vitamin B1 and, in the form of the co-factor thiamine pyrophosphate (TPP), plays a role in important metabolic processes such as carbohydrate metabolism. It has now been found that the combination of thiamine

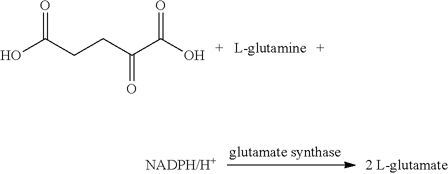

g an important role in this regard.

First, there is a synergy in the administration of thiamine and taxifolin after, before, or during alcohol consumption, in particular with regard to alcohol-related consequential conditions. For this, the effect of thiamine or TPP, respectively, as a component of the α-ketoglutarate dehydrogenase enzyme complex in combination with the effect of the taxifolin is responsible.

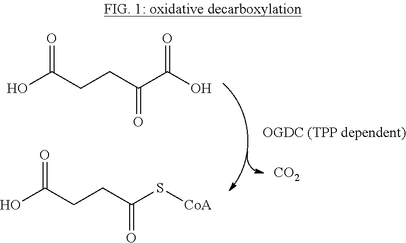

This is the case because the α-ketoglutarate dehydrogenase enzyme complex (OGDC) catalyzes the oxidative decarboxylation of α-ketoglutarate to succinyl-CoA by cleaving off CO₂ as a part of the citrate cycle.

If the level of thiamine or TPP, respectively, is too low, this process proceeds less efficient and results in an accumulation of α-ketoglutarate (AKG), which also occurs in the nervous tissue in the astrocytes. AKG is now increasingly metabolized by the enzyme glutamate synthase to the neurotransmitter L-glutamate, which consequently also accumulates in increased concentration, including in the CNS.

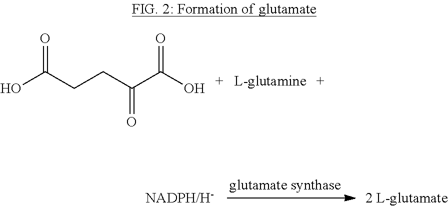

Alcohol-related consequential conditions as well as alcohol-related nerve damage are directly related to a reduction in GABA_{A} receptor density during alcohol consumption and the associated overexcitation of neurons (rebound) after alcohol breakdown. Since the excitatory neurotransmitter glutamate counteracts the inhibitory effect of the neurotransmitter GABA, this effect is further enhanced by an increased glutamate concentration. This leads to an overexcitation of the nerve cell, whereby on the one hand cell death can occur due to excitotoxicity, and on the other hand negative consequential conditions can also occur (“hangover symptoms”). Therefore, a combination of thiamine and taxifolin is particularly nutritionally advantageous for this application.

Furthermore, the combination of taxifolin and thiamine is advantageous because, surprisingly and for the first time, it was found that thiamine can reduce oxidized taxifolin, thus enhancing and prolonging the effect of the flavonoid in vivo. The oxidation of taxifolin initially occurs at the unstable catechol group with the formation of an orthoquinone; as a result, the flavonoid loses its physiological effect.

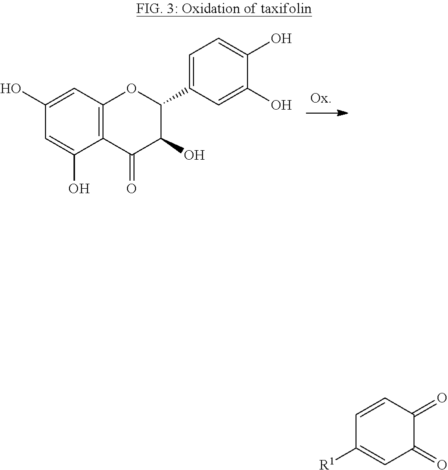

Thiamine is now able to effectively reduce the oxidized orthoquinone group to the active flavonoid taxifolin with a catechol group in vivo. For this, thiamine is first converted by hydroxide ions to the thiol form by opening of the thiazole ring, which then reduces the orthoquinone by formation of a disulfide bridge. This can counteract oxidation of the flavonoid, which increases efficacy.

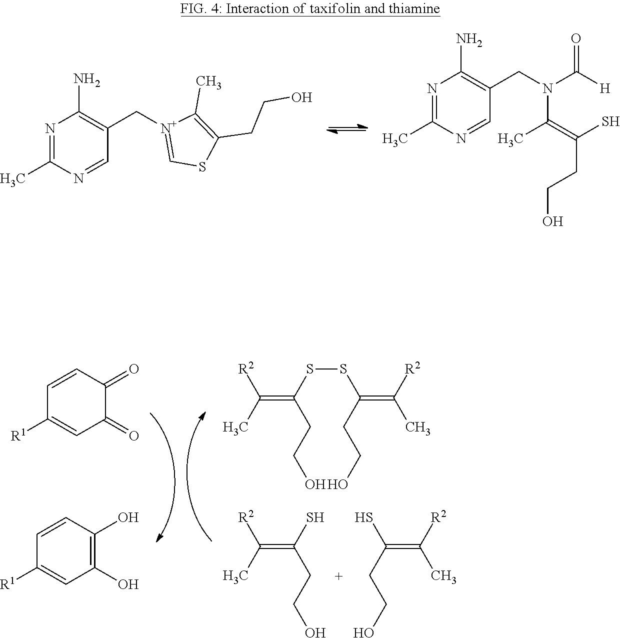

This is surprising, since thiols typically add to orthoquinones by Michael addition and would thus render the flavonoid definitively ineffective. This conjugation could, for example, be increasingly observed for the amino acid L-cysteine and the tripeptide glutathione, which were not as effective in reducing oxidized taxifolin and thereby prolonging the effect in vivo.

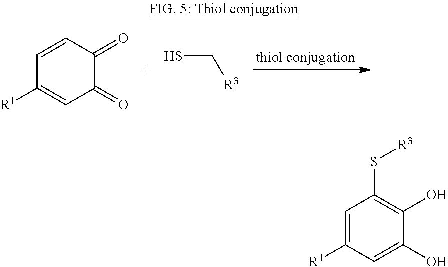

Furthermore, it was found that thiamine is not capable of effectively reducing oxidized flavonoids with a double bond at position 2,3 such as quercetin. To these oxidized flavonoids/orthoquinones, the thiol form of thiamine also increasingly adds via Michael addition, which is the reason why the efficacy of these flavonoids and of thiamine in combination is actually even reduced in vivo.

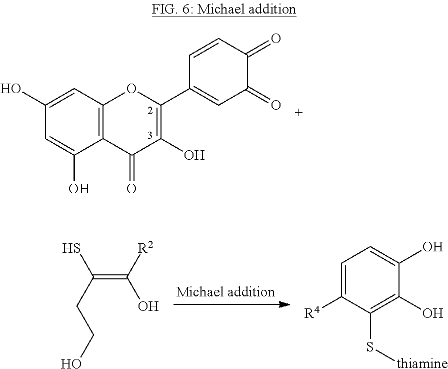

Therefore, this synergy between taxifolin and thiamine is surprising and unusual for the substance class of flavonoids. The combined intake of these two active substances during alcohol consumption is very advantageous.

However, the redox reaction between thiamine and taxifolin can also occur unintentionally during time of storage, which results in reduction of the thiamine content of the preparation and has a negative effect on the best-before date. In order to ensure maximum storage stability, various galenic formulations of the flavonoid have been prepared, including the formulation of solid dispersions with typical pharmaceutical polymers such as polyvinylpyrrolidone (PVP), polyvinylpyrrolidone vinyl acetate copolymer, polyacrylic acids, as well as various biopolymers such as hydroxypropylmethylcellulose, hydroxypropylcellulose, methylcellulose, sodium carboxymethylcellulose, maltodextrin, shellac, collagen hydrolysate, chitosan, gellan, xanthan and alginate. Moreover, the formulation of co-crystals with urea, caffeine and nicotinamide, the formulation of micelles with various surfactants such as lecithin, polysorbate 80, vitamin E TPGS, macrogol-15-hydroxystearate, macrogol glycerol hydroxystearate and sodium dodecyl sulfate were also carried out on a laboratory scale, although in each case no sufficient improvement in stability was observed when mixed with thiamine.

Therefore, a formulation with an excipient is required for a successful combination of thiamine and taxifolin.

Surprisingly, only two flavonoid formulations were found to be effective in preventing unwanted interactions between taxifolin and thiamine. One is a) the complex formation of taxifolin with cylcodextrins, in particular with β-cyclodextrin (E459), and the other is b) the formulation of the flavonoid into a solid dispersion in basic polymethacrylates, in particular in basic methacrylate copolymers approved for food use (E1205), for example Eudraguard® protect.

It is therefore an object of the present invention to provide a formulation for oral administration comprising.
(i) Taxifolin or a pharmaceutically acceptable salt, derivative or prodrug thereof,
(ii) thiamine or a pharmaceutically acceptable salt, derivative or prodrug thereof, and
(iii) at least one excipient selected from a) β-cyclodextrin and derivatives thereof, and b) a basic (co)polymer of methacrylic acid and/or methacrylate,
   wherein taxifolin is present (a) as a complex with the β-cyclodextrin or (b) as a solid dispersion with the basic (co)polymer of methacrylic acid and/or methacrylate.

In a first embodiment of the present invention, taxifolin is present in the form of an inclusion complex with β-cyclodextrin. The complex formation increases the solubility and dissolution of taxifolin and substantially improves its biological activity. In particular, however, the unstable catechol group of taxifolin is entrapped and thus protected from oxidation, as evidenced by ¹H-NMR and FT-IR spectroscopies. This prevents the formation of an orthoquinone group by oxidation of taxifolin during storage, which also prevents breakdown of thiamine by forming a disulfide bridge. Contrary to expert opinion, it was found that only β-cyclodextrin, but not γ-cyclodextrin, is able to entrap the catechol group. Furthermore, by DSC measurements and addition of urea, it was found that γ-cyclodextrin tends to form supramolecular complexes and precipitates after initially good dissolution behavior. β-cyclodextrin is preferably used in a molar ratio of β-cyclodextrin:taxifolin of 0.5:2 to 2:0.5, preferably in a ratio of 0.8:1 to 1.5:1. A molar ratio of β-cyclodextrin:taxifolin of about 1:1 is particularly preferred. The use in the form of a taxifolin/β-CD inclusion complex formed by spray drying is particularly preferred.

β-Cyclodextrin (β-CD) is a cyclic oligosaccharide that is composed of seven α-1,4-glycosidically linked glucose molecules. It may be present in an underivatized or derivatized form in a formulation according to the present invention, in which, for example, one or more hydroxyl groups of glucose units carry substituents. For example, the C6 carbon atom may be alkoxylated or hydroxyalkylated on one or more glucose units of the β-cyclodextrin. For example, the hydrogen atom of the hydroxyl group may be replaced by C1-18 alkyl or C1-18 hydroxyalkyl groups on the C6 carbon atom of one or more glucose units. Particularly preferred are 2,6-di-O-methyl-cyclodextrin and 2-hydroxypropyl-cyclodextrin. Furthermore, sulfoalkyl cyclodextrins, in particular sulfoethyl-, sulfopropyl- and sulfobutyl-β-cyclodextrin are of interest.

In order to improve complex stability, a formulation according to the present invention comprising β-cyclodextrin and taxifolin may further contain one or more water-soluble polymers. This can effectively prevent recrystallization of the active substance taxifolin and thus maintain the high initial concentration for a long time. For this purpose, very low polymer concentrations are often sufficient to achieve the desired effect. The water-soluble polymer is preferably present in solution in an amount of at least 0.0025% w/v, in particular 0.0025-1.0% w/v, further preferably 0.025-0.5% w/v, for example 0.25% w/v. With reference to the taxifolin, the polymer:flavonoid mass ratio is preferably between 1:0.5 and 1:80, in particular between 1:3 and 1:15. Mass ratios in the range between 1:6 and 1:8 have been found to be optimal in practice.

Examples of water-soluble polymers particularly suitable according to the present invention are polyethylene glycol, e.g. PEG 6000, polyvinyl alcohol, poloxamer, e.g. Poloxamer 188 and mixtures thereof, such as mixtures of PEG and PVA (Kollicoat® IR). These polymers are composed of ethylene oxide blocks and show very promising properties. The interactions with the hydroxy groups of taxifolin are not so strong that precipitation occurs, and at the same time the polymers also interact with the hydroxy groups of β-cyclodextrin. This increases the complex stability.

The increase in complex stability can be explained by the fact that the polymer interacts with the active substance and the β-cyclodextrin, and thereby stabilizes the active substance in the cavity of the cyclodextrin (ternary complex). This must be taken into consideration when selecting the appropriate polymer, because if the interaction with the active substance is too strong, the polymer-active substance complex flocculates and Ks decreases. If the interaction with the cyclodextrin is too strong, the polymer and active substance will compete for the CD cavity and Ks will also decrease. Finally, it is important ensure that the polymer must not increase or must only slightly increase the viscosity of the solution, since otherwise CD complex formation will be aggravated.

In order to improve the dissolution behavior as well as the stability, a formulation according to the present invention with β-cyclodextrin or a basic (co-) polymer of methacrylic acid and/or methacrylate and taxifolin may further contain choline salts/(2-hydroxyethyl)-trimethylammonium salts. In experiments, these compounds, such as choline chloride, choline bitartrate or choline citrate, have surprisingly proven to be helpful additives. Formulations containing choline cations showed both faster dissolution, lower recrystallization and higher overall solubility. This is due to two mechanisms:

Choline cations interfere with the formation of hydrogen bonds due to the quaternary alkyl ammonium group in solution, and accordingly reduce hydrophobic effects. As a result, less hydrophilic substances dissolve more easily or do not precipitate out of a supersaturated solution (“salting in effect”). Specifically, it was found for the first time that only the addition of choline cations results in better dissolution behavior of taxifolin formulations, with this being due to faster dissolution as well as reduced recrystallization. In addition, choline cations are able to form ternary complexes with taxifolin/β-cyclodextrin complexes, thus increasing complex stability. This dual mechanism could only be found for the choline cation.

Choline compounds are preferably used in a taxifolin:choline mass ratio of 5:1 to 1:20, based on the pure mass of the choline cation. A ratio of 2:1 to 1:2.5 has proven to be particularly advantageous, the optimum being 1:0.85. All salts of the choline cation can be used as choline compounds, with compounds having organic, multi-proton acid anions (choline bitartrate or choline bitartrate) being preferred because of their acidic effect. This keeps the concentration of hydroxide ions necessary for the opening of the thiazole ring low during storage, which can further reduce the breakdown of thiamine by oxidation.

In addition, choline compounds have an important function in the triglyceride metabolism of liver cells, with a deficiency of choline leading to increased production of triglycerides. Since the metabolism of ethanol occurs via the enzymes alcohol dehydrogenase (ADH) as well as aldehyde dehydrogenase (ALDH) by consumption of NAD+, various NAD+-dependent processes—such as β-oxidation—are inhibited by alcohol consumption. This leads to a reduced consumption of triglycerides, which can result in the development of disease patterns such as alcoholic fatty liver. Therefore, the uptake of choline is advantageous in preventing further accumulation of triglycerides. It has now been found that this effect can be enhanced by the addition of taxifolin formulations as well as thiamine. This is initially due to an inhibition of the enzyme diacylglycerol-O-acyltransferase (DGAT) by taxifolin, whereby in the final step of triglyceride metabolism no fat molecule is formed from diacylglycerol but, together with a choline compound, phosphatidylcholine, which as a cell membrane component does not contribute to the development of fatty liver. The effect of taxifolin can now be enhanced by the redox reaction with thiamine. In addition, the use of taxifolin as a β-cyclodextrin complex or as a solid dispersion in basic polymethacrylate is particularly advantageous in order to minimize breakdown of taxifolin and to ensure optimal release, stability, and water solubility. The use of taxifolin in the form of a β-cyclodextrin complex or as a solid dispersion in basic polymethacrylate is also of great importance in order to ensure storage stability in combination with thiamine. Therefore, the use of choline compounds for the treatment and prevention of alcohol-related liver diseases and liver damage in combination with thiamine and taxifolin (in the form of a β-cyclodextrin complex or as a solid dispersion in basic polymethacrylate) is particularly advantageous.

In a second embodiment of the present invention, a solid dispersion with basic polymers or copolymers of methacrylic acid and/or methacrylate is present. In this way, good water solubility and high bioavailability of the taxifolin is achieved. Examples of suitable polymethacrylates are Eudragit® E, Eudraguard® protect or Kollicoat® Smartseal.

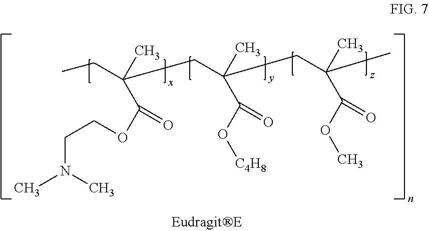

The observed improvement in solubility is due to the intermolecular interactions between the carbonyl group of the methacrylic ester and the hydroxy groups (or similar groups) of the taxifolin. This stabilizes the taxifolin in its amorphous form, which significantly improves water solubility. Unlike other polymers such as PVP, the cationic aminoalkyl groups of Eudragit, which are cationic when in protonated state, make the polymer water-soluble, even when it strongly interacts with the taxifolin.

Further, by forming of a solid dispersion of taxifolin in basic (co)polymer of methacrylic acid and/or methacrylate polymethacrylate, unwanted interactions between taxifolin and thiamine can be prevented. This is due to the fact that taxifolin enters into ionic interactions with these polymers, in particular between the aminoalkyl residue of the polymer and the hydroxy groups of the catechol group of the flavonoid, as could be demonstrated by FT-IR spectroscopy. This can also prevent the formation of an orthoquinone group by oxidation of the taxifolin during storage, which also avoids breakdown of the thiamine by forming the disulfide bridges. These ionic interactions could not be found for any other polymer, therefore the other polymers did not have any significant effect on the interactions between thiamine and taxifolin. Preferred weight ratios between taxifolin and basic (co)polymer of methacrylic acid and/or methacrylate are in the range of 1:1 to 1:3, particularly preferably about 1:2. Preferably, the solid dispersion is prepared by melt extrusion of the polymer with the flavonoid or by dissolution of the polymer and the flavonoid in a common solvent, such as ethanol or acetone, and subsequently removing the solvent e.g. by spray drying.

Surprisingly, in order to further prevent interactions between thiamine and taxifolin during storage, a microencapsulation of the thiamine has proven to be very useful. Various coating materials are available for this purpose, for example hydrogenated lipids, e.g. from vegetable oils such as palm oil, carnauba wax or beeswax, cellulose derivatives such as ethyl cellulose, gum arabic, fatty acids, di- and monoglycerides, starch or starch derivatives as well as polymethacrylates. Hydrogenated palm oil lipids, carnauba wax, fatty acids, di- and monoglycerides, acid/neutral polymethacrylates as well as ethyl cellulose have proven particularly suitable. Thereby, breakdown of the thiamine with formation of the disulfide bridges during storage is prevented.

Furthermore, since taxifolin inhibits the resorption of thiamine by interaction with the intestinal thiamine transporters in the bowels, the development of a suitable galenic in order to solve this problem was also part of the present invention. It has been shown that an instant-release formulation of taxifolin in combination with an extended-release formulation of thiamine leads to optimal absorption of both drugs. This is because while the flavonoid is present in the stomach in dissolved state within minutes of administration and is resorbed in the anterior regions of the GI tract, the thiamine is resorbed in a delayed manner over a longer period of time and in posterior regions of the GI tract, which does not cause any negative interactions.

It has been found that the best way to instant-release formulate taxifolin is to form an inclusion complex with β-cyclodextrin or to form a solid dispersion in basic polymethacrylates. The best option for extended-release formulation of the thiamine is microencapsulation, particularly with hydrogenated palm oil lipids, carnauba wax, fatty acids, di- and monoglycerides, neutral/acidic polymethacrylates, and ethyl cellulose as the coating materials.

Thiamine is preferably used in isolated form as thiamine mononitrate or thiamine hydrochloride. Thiamine hydrochloride is particularly preferred, since it has been shown in experiments that the nitrate group, by forming nitrite, can oxidize taxifolin to orthoquinone, which in turn leads to the breakdown of the thiamine. On the other hand, the chloride ions are inert and thus preferred. Since the reduction of nitrate to nitrite is dependent on the pH and occurs increasingly in the acidic environment of the stomach, an extended-release formulation is particularly advantageous for thiamine nitrate.

Taxifolin can optionally be used in the form of pharmaceutically acceptable salts, derivatives or prodrugs, especially with glycosyl, ether or ester groups at the positions of OH groups. Examples of glycosides are monosaccarides and oligosaccharides. Suitable ethers include, in particular, alkyl ethers, aryl ethers and hydroxyalkyl ethers. Suitable esters include, for example, carbonates, carbamates, sulfamates, phosphates/phosphonates, neutral or anionic carboxylic acid esters, and amino acid esters. These derivatives are converted back to the main active substance taxifolin by enzymatic cleavage in the body.

According to the present invention, mono- and oligoglycosyl residues preferably comprise hexosyl residues, in particular ramnosyl residues and glucosyl residues. Further examples of suitable hexosyl residues include allosyl, altrosyl, galactosyl, gulosyl, idosyl, mannosyl and talosyl. Alternatively or additionally, mono- and oligoglycosyl residues may comprise pentosyl residues. The glycosyl residues may be α- or β-glycosidically linked to the main body. For example, a preferred disaccharide is 6-O-(6-deoxy-α-L-mannopyranosyl)-β-D-glucopyranoside.

In addition, it is possible to convert the phenolic hydroxyl group of taxifolin into a hemiacetal with various aldehydes (e.g., acetaldehyde). The hydroxy group of this hemiacetal can now be derivatized in the same way as the phenolic hydroxy group. An example of this is the phosphonooxy alkyl prodrugs.

Taxifolin is preferably used in the form of an extract from crushed larch wood, since high concentrations of this flavonoid are found in said wood, especially in the tree stumps. In addition, other flavonoids are present in comparatively high concentrations, which can also be effectively reduced by thiamine. Aromadendrin and eriodictyol are of particular interest in this context. Like taxifolin, these flavonoids are characterized by a single bond at position 2,3. Extract from larch wood is clearly preferred because, unlike most plant extracts which would also contain taxifolin, it has only a very small proportion of flavonoids with a double bond at 2,3, such as quercetin. Preferably, an extract of Dahurian larch (Larix gmelinii) is used, which can be obtained by ethanol-water extraction and has a taxifolin content of at least 88%, preferably a purity between 90% and 97%, and most preferably a purity of 90%-93%. This is important because only with a sufficiently high taxifolin content the formulation as β-cyclodextrin complex or solid dispersion in basic polymethacrylates can be carried out efficiently. The branded extracts Lavitol® from Ametis JSC and Flavit® from Balinvest Ltd. have proven to be particularly preferred.

The total taxifolin dosage can altogether be in the range from 10 mg to 500 mg (preferably 30-400 mg, particularly preferably 50-150 mg, optimally 100 mg). The thiamine dosage may be in the range from 0.1 mg to 250 mg (preferably 1-100 mg, more preferably 5-50 mg, optimally 10 mg). The total dosage may be divided into several dosage units.

Ratios between taxifolin:thiamine of 700:1 to 1:1 have proven useful, in particular between 100:1 and 3:1. The best ratio is in the range between 20:1 and 5:1, whereby a ratio in the range of 10:1 is the optimum range.

The formulation according to the present invention for oral administration may further comprise one or more pharmacologically acceptable excipients and/or carriers, and/or one or more further ingredients.

Examples of further ingredients include vitamins (in particular B vitamins) as well as their pharmaceutically acceptable salts, derivatives and prodrugs, for example of the vitamins riboflavin, niacin, pantothenic acid, pyridoxine, biotin, folic acid, cobalamin, ascorbic acid, retinol, cholecalciferol, tocopherol, phylloquinone. In addition, various minerals and trace elements, as well as their pharmaceutically acceptable salts and complexes, may also be included, for example, of calcium, magnesium, potassium, sodium, chromium, copper, manganese, molybdenum, selenium, zinc, cobalt, silicon, iodine and fluorine. Finally, further vitaminoids, as well as their pharmaceutically acceptable salts, derivatives and prodrugs may be included, for example of choline, coenzyme Q10 (ubiquinone-10), L-carnitine, as well as various amino acids, their pharmaceutically acceptable salts, derivatives and prodrugs, for example of glycine, L-proline, L-tyrosine, L-glutamine, L-cysteine, L-asparagine, L-arginine, L-histidine, L-isoleucine, L-leucine, L-lysine, L-methionine, L-phenylalanine, L-threonine, L-tryptophan, L-valine, L-alanine, L-aspartic acid, L-glutamic acid and L-serine.

The formulation according to the present invention is designed to be administered orally. The formulation may be in the form of powder, granules, capsules, tablets, chewable tablets, effervescent tablets, coated tablets, sachets or solutions/suspensions for oral administration, and the total amount of the dosage may be divided into several dosage units. Particularly preferred is the dosage form in the form of compressed tablets, film-coated tablets, chewable tablets as well as effervescent tablets.

In the preparation of the formulation, suitable excipients can be used which can be mixed with the active substances of the composition, in particular polyethylene glycol, polyvinyl alcohol, silicon dioxide, starch derivatives such as maltodextrin, potato starch or sodium starch glycolate (Explotab®), metal stearates such as magnesium stearate, surfactants such as lauryl sulfate, titanium dioxide, carbonates, sugars and sugar alcohols, talc, cellulose derivatives such as hydroxypropyl cellulose, microcrystalline cellulose, methyl cellulose or carboxymethylcellulose, and other excipients and additives known to the skilled person. The composition may be mixed, granulated and/or compressed in a conventional manner, or tableted/compressed in tablet form, wherein the tablet is preferably coated with a film (film-coated tablet). The preparation of such formulations can be carried out in the usual manner that is familiar to the person skilled in the art.

In addition to the active substances, solid formulations for oral administration may contain common excipients and carriers, such as diluents, e.g., lactose, dextrose, sucrose, cellulose, corn starch or potato starch; lubricants, e.g., silicate, talc, stearic acid, magnesium or calcium stearate and/or polyethylene glycols; binding agents, e.g. starches, gum arabic, gelatin, methylcellulose, carboxymethylcellulose, or polyvinylpyrrolidone; disintegrants, e.g., starch, alginic acid, alginates, or sodium starch glycolates, foaming mixtures; colorants; sweeteners; wetting agents, such as lecithin, polysorbates, lauryl sulfates; and other common formulation adjuvants.

Liquid formulations for oral administration may be, for example, dispersions, syrups, emulsions, and suspensions. For example, a syrup may contain sucrose or sucrose with glycerol and/or mannitol and/or sorbitol as a carrier. Suspensions and emulsions may contain as carriers, for example, a natural resin, agar, sodium alginate, pectin, methylcellulose, carboxymethylcellulose or polyvinyl alcohol.

The formulations according to the present invention may be used for the prevention and/or treatment of alcohol intoxications, consequential conditions and diseases associated with alcohol consumption, or alcoholism.

The term “alcoholism” as used herein includes physical and/or psychological dependence on alcohol (addiction syndrome). It has been found that administration of a formulation according to the present invention can counteract the development of an addiction syndrome and thus can be used to prevent alcoholism. In cases of already existing alcoholism, it is possible to provide treatment by using a formulation according to the present invention, including alcohol dishabituation and/or alcohol withdrawal.

Withdrawal symptoms may occur when alcohol consumption is reduced or abruptly stopped. Withdrawal symptoms include nausea, nervousness, sleep disturbances, an urge to drink alcohol, irritability, and depression. If the physical dependence is in advanced state, sweating, tremors, flu-like symptoms, seizures and hallucinations occur. By using the formulation according to the present invention, these and other withdrawal systems can be prevented or mitigated.

As used herein, the term “alcohol intoxication” comprises all stages of acute alcohol intoxication. Depending on the blood alcohol concentration, a distinction is made between the stages of excitation (0.2-2.0‰), hypnosis (2.0-2.5‰), narcosis (2.5-4.0‰), and asphyxia (above 4.0‰). Due to their specific binding at the α4β3δ or α6β3δ GABA_{A} receptor, flavonoids of formula (I) are capable to act as allosteric modulators to counteract the binding of alcohol at the GABA_{A} receptor, and thus render it ineffective.

In addition to preventing and treating acute alcohol intoxications, the formulations disclosed herein can also be used in accordance with the present invention to prevent and/or treat consequential conditions associated with alcohol consumption and to prevent secondary diseases. Such secondary diseases are diseases attributable to long-term alcohol abuse, such as, in particular, impairments of the nervous system (due to destruction of axons such as the myelin sheaths of the brain and the peripheral nervous system, e.g., neuropsychological weaknesses, memory disorders, impaired consciousness, dementia syndrome, neuropathic pain, etc.) and, in particular, liver damage.

Consequential conditions that are associated with alcohol consumption further include acute consequences, such as hangovers in particular. In this context, a hangover is understood as the feeling of being unwell and the impairment of physical and mental performance as a result of excessive alcohol consumption.

A hangover primarily comprises the symptoms of headache, stomach pain, nausea and vomiting, difficulty concentrating, increased tendency to sweat, stomach and muscle pain, depressed mood, and a general feeling of malaise on subsequent days, especially the day after excessive alcohol consumption.

By using the formulations described herein, the present invention succeeds in reducing the frequency of alcohol consumption compared to the frequency before treatment. Similarly, it succeeds in reducing the amount of alcohol consumed. Furthermore, it succeeds in increasing the abstinence rate.

Preferably, the formulation according to the present invention is administered orally in tablet form. The application of administering the preparation according to the present invention can take place before, during or after alcohol consumption. Preferably, administration is 30 min to 120 min before the start of alcohol consumption. Administration of the preparation according to the present invention along with a (high-fat) meal has been shown to be advantageous.

The present invention will be further illustrated by the following figures and examples which are not intended to limit the subject-matter of the claims.

### FIGURES

FIG. 1a ¹H-NMR-Spectres of taxifolin complexes with various cyclodextrins

FIG. 1b relevant overlays

FIG. 1c Assigning the peaks of the spectres to the various taxifolin protons

FIG. 2: Labeled dissolution graph showing the dissolution behavior of cyclodextrin complexes
3: Taxifolin/β-CD complex
2: Eudragit® E solid dispersion
1: Taxifolin (reference)

FIG. 3: Thin layer chromatographic separation of various compositions with taxifolin and thiamine

### EXAMPLES

### 1. H¹-NMR Spectrometric Study of Various Cyclodextrin Complexes

In order to qualitatively detect the complex formation in aqueous solution, ¹H NMR spectroscopy was used. This allows the characteristic spectra of taxifolin and the cyclodextrin to be determined. When a complex is formed, a shift of certain signals occurs. In addition, the exact three-dimensional structure of the complex and the conformation of the flavonoid in the cyclodextrin cavity can be determined.

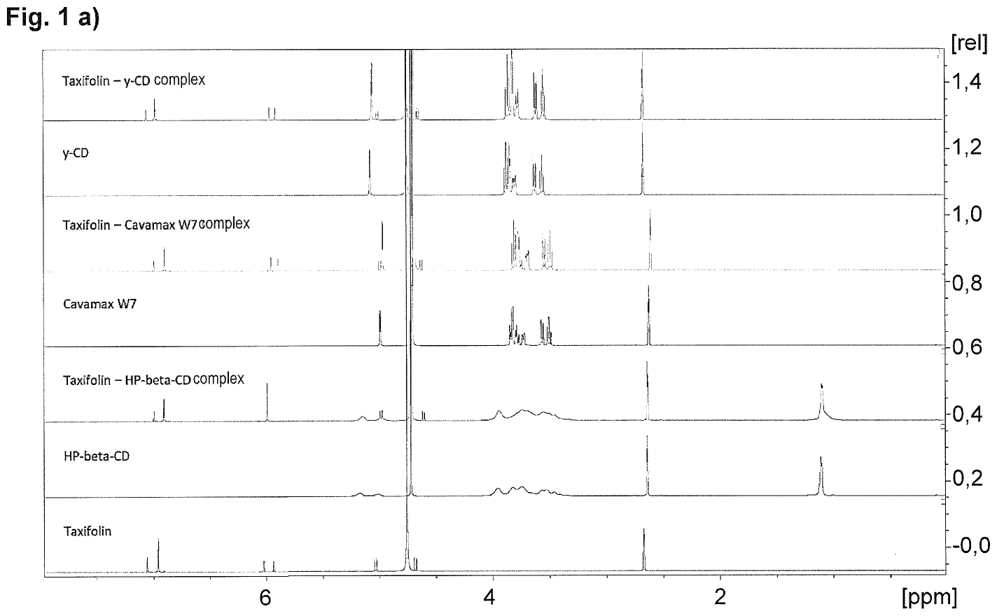

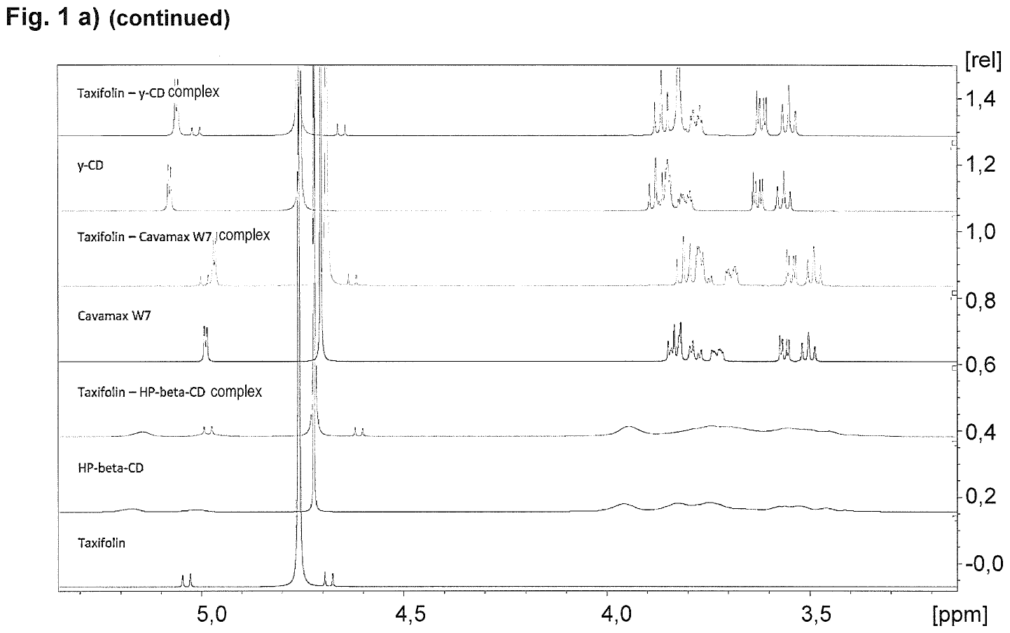

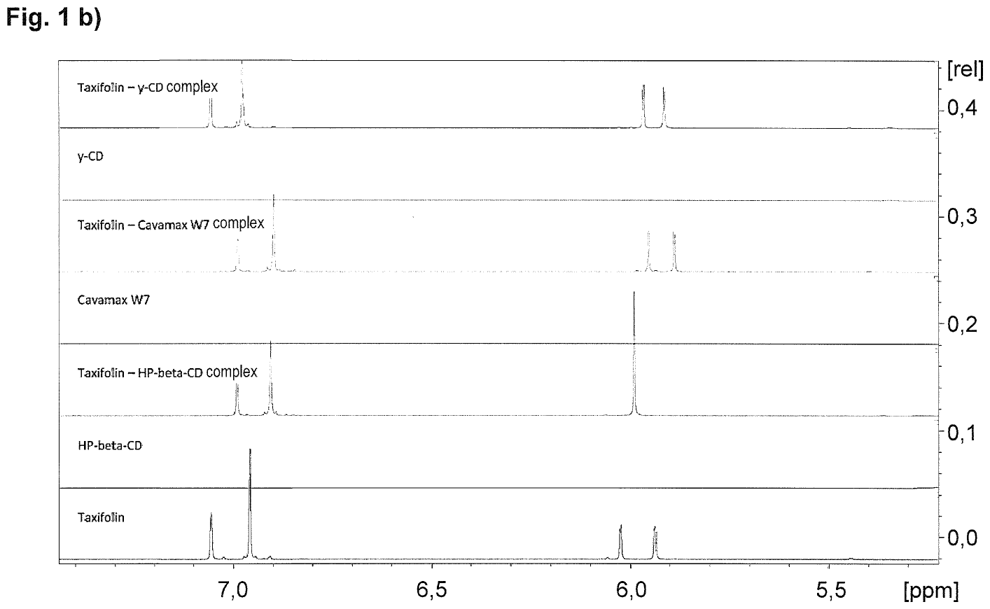

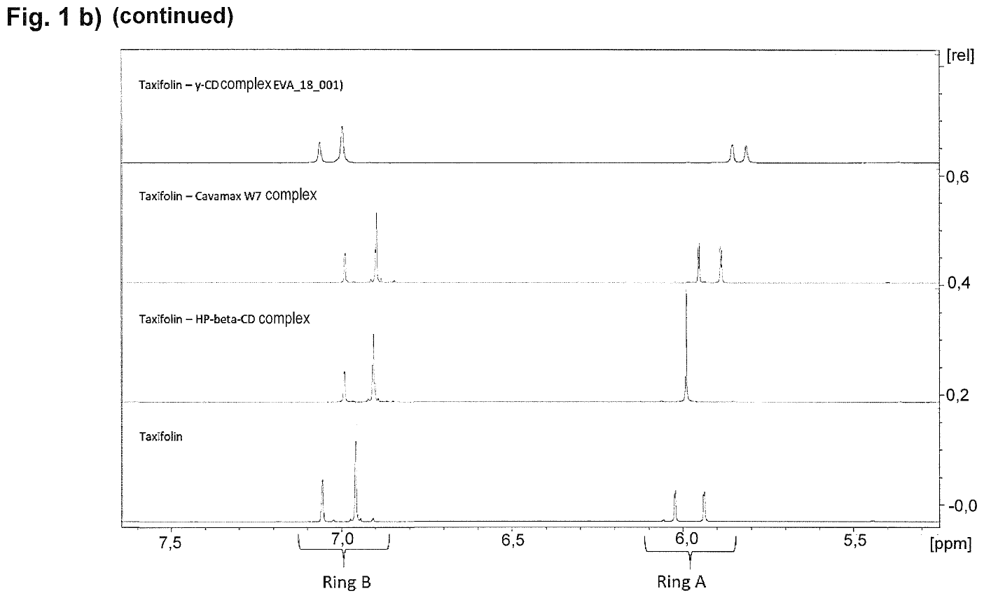

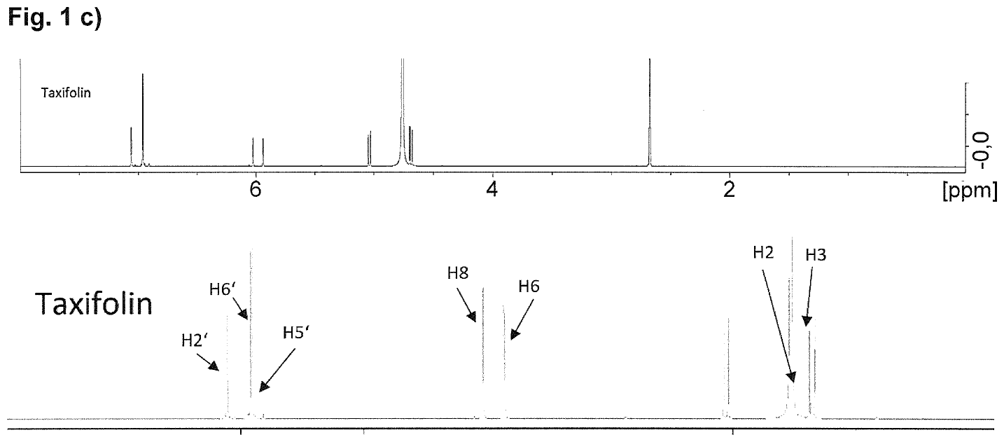

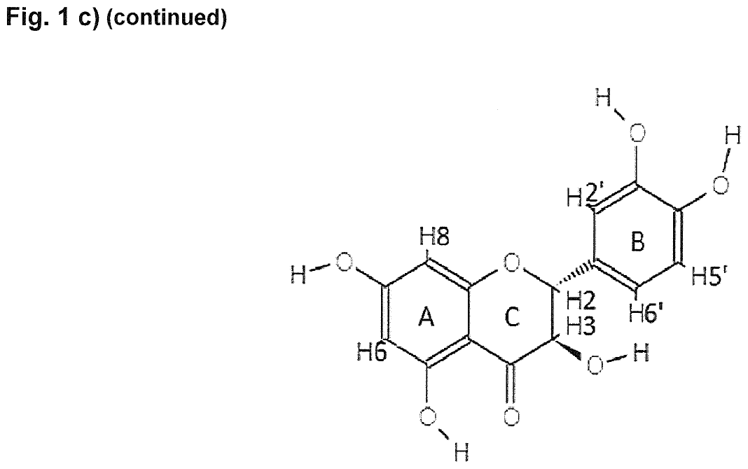

In order to achieve complex formation in solution, taxifolin and the respective cyclodextrin (β/CAVAMAX W7, HP-β or γ) were weighed at a molar ratio of 1:1, dissolved in D₂O/DMSO (80/20 v/v) and stirred for 3 h at room temperature and 600 rpm. In the following, the sample was measured. The reference solutions (taxifolin, β-CD, HP-β-CD and γ-CD) were dissolved in D₂O/DMSO (80/20 v/v) only and then measured. The results are shown in FIG. 1.

Discussion: Due to the signal shifts, the results clearly indicate complex formation in solution. However, the results can also be used to accurately predict the location of the flavonoid in the CD cavity. This is because the protons, which exhibit a signal shift due to complex formation are embedded in the CD cavity. Here, there are clear differences between β-CD/HP-β-CD and γ-CD.

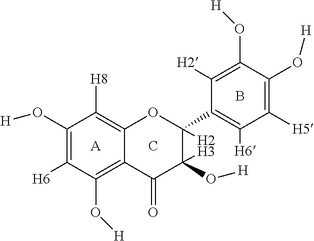

In β-CD and HP-β-CD, the signals of the protons H2′, H5′ and H6′ are shifted, which indicates that ring B is embedded in the CD cavity. This is also consistent with the prevailing view that β-CDs mainly include monocyclic aromatics because of their ring size. Based on ¹H-NMR spectroscopy, the following conformation of the flavonoid in the β-CD/HP-β-CD cavity can be predicted:

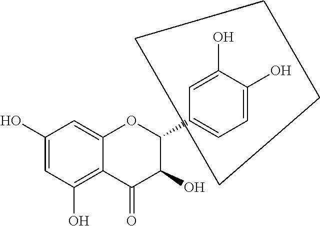 [SMILES: O=C1c2c(O)cc(O)cc2O[C@H](c2ccc(O)c(O)c2)[C@H]1O]

However, it is interesting to note that in the HP-β-CD complex, the signals of the protons H6 and H8 combine to form a common peak. This is probably due to hydrogen bonding between the hydroxypropyl residue of cyclodextrin and various residues on ring A.

In γ-CD, in particular the signals of protons H6 and H8 are shifted, however, although less pronounced, also those of protons H2 and H3 are shifted. This indicates that rings A and C in part are embedded in the CD cavity. This is also consistent with the prevailing view that γ-CD mainly includes polycyclic aromatics due to the ring size. Based on ¹H-NMR spectroscopy, the following conformation of flavonoid in the γ-CD cavity can be predicted:

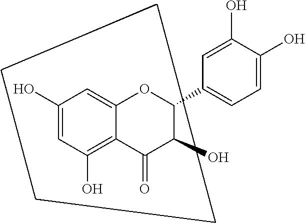 [SMILES: O=C1c2c(O)cc(O)cc2O[C@H](c2ccc(O)c(O)c2)[C@H]1O]

The different position of the flavonoid in the CD cavity naturally influences the interactions of the flavonoid with thiamine. Only a complex with β-cyclodextrin can prevent undesired redox reactions during storage time, whereas γ-cyclodextrin has no influence on this.

### 2. Preparation of Cyclodextrin/Taxifolin Complexes

Different methods to prepare the complexes were examined and compared:

### Spray Drying β-CD (SD β).

10000 mg of taxifolin and 37300 mg of β-cyclodextrin were each weighed out in a molar ratio of 1:1 and placed in a shared beaker. Correspondingly 940 ml of distilled water (25° C., 5% w/v) was added to the β-CD-taxifolin mixture now, followed by stirring at 25° C. for 30 min with a high-shear mixer (3000 min-1) until a concentrated suspension was formed. This suspension was stirred for 24 h at 600 rpm and 25° C. in the absence of oxygen to complete the complex formation. The solution was vacuum filtered (0.45 μm membrane filter) to remove undissolved flavonoid and cyclodextrin residues, and the filtrate was then spray dried.

Parameters: V=900 ml, T(in)=125° C.; pump rate: 20%; aspirator: 100%, spray gas: 55 mm; T(out)=71° C.

### Freeze Drying β-CD (FD β).

1000 mg of taxifolin and 3730 mg of β-cyclodextrin were each weighed at a molar ration of 1:1 and placed in a shared beaker. Accordingly, to the β-CD/taxifolin mixture (5% w/v) 94 ml distilled water was then added and stirred for 30 min at 30° C. with a homogenizer (3000 min-1) until a suspension was formed. This suspension was stirred for 24 h at 600 rpm and 25° C. in the absence of oxygen to complete the complex formation. The solution was vacuum filtered (0.45 μm membrane filter) to remove undissolved flavonoid and cyclodextrin residues, and the filtrate was then cooled to −80° C. for 24 h in centrifuge tubes to freeze it. In the following, the tubes were placed in the freeze dryer and the pressure was adjusted to 0.05 mbar and the temperature was adjusted to −30° C. Under these conditions, the solution was freeze dried for 96 h.

### Freeze Drying γ-CD (FD-γ).

1000 mg of taxifolin and 4266 mg of γ-cyclodextrin were each weighed out at a molar ratio of 1:1 and placed in a shared beaker. Accordingly, 265 ml distilled water (2.5% w/v, 25° C.) was then added to the γ-CD-taxifolin mixture and stirred for 30 min with a homogenizer (3000 min-1) until a clear solution was formed. The solution was vacuum filtered (0.45 μm membrane filter) to remove undissolved flavonoid and cyclodextrin residues, and the filtrate was then cooled to −80° C. for 24 h in centrifuge tubes to freeze it. In the following, the tubes were placed in the freeze dryer and the pressure was adjusted to 0.05 mbar and the temperature was adjusted to −30° C. Under these conditions, the solution was freeze-dried for 96 h.

Phys. Mix 1:1 β-CD and γ-CD, Respectively.

Taxifolin and β-cyclodextrin or γ-cyclodextrin were weighed out at a molar ratio of 1:1 and mixed together in a mortar.

### 3. DSC Analyses of the Cyclodextrin Complexes

In order to be able to quantitatively determine the efficiency of the encapsulation method, various measurement methods are available. One very popular method is Differential Scanning calorimetry (DSC), which can be used to determine the residual content of the free active substance on the basis of characteristic endothermic peaks (approx. 240° C. for taxifolin). Since the active substance/cyclodextrin complex has a different decomposition or melting point, the absence of the “active substance peak” can thus be used to indirectly infer high encapsulation efficiency.

Therefore, the comparison of the sample peaks with the peaks of the pure active substance, the pure cyclodextrin and an equimolar physical mixture (Phys. Mix 1:1) is of particular importance. The latter serves as a reference for the samples, since in a physical mixture the drug is present in its free, uncomplexed form (encapsulation efficiency=0%). A complete absence of the drug peak at 240° C. corresponds to an encapsulation efficiency of 100%. Based on the area of the characteristic drug peak of the individual samples, they can be compared with each other and with the physical mixture. The main advantage of this measurement method is, on the one hand, the quite high precision and, above all, the possibility of measuring the samples in solid state. This prevents the complex equilibrium from being influenced or readjusted by water or other solvents.

In the case of the β-cyclodextrin samples SD β and FD β, characteristic drug peaks cannot be detected any more. Moreover, the intensity of the broad endothermic peak decreases significantly between 70° C. and 100° C. compared to the reference samples (Phys. Mix 1:1). This indicates that less water escapes from the β-cyclodextrin cavity during heating as it is occupied by the flavonoid. Therefore, from the DSC thermograms, it appears that in these samples the flavonoid is completely present as a β-CD complex and the encapsulation efficiency is close to 100%.

The thermogram of the γ-CD complex is fundamentally different from the thermograms of the β-CD complexes. Although the γ-CD complex sample does also not have a characteristic drug peak that coincides with the physical mixture (Phys. Mix. 1:1). This indicates complete encapsulation, as no free flavonoid can be detected anymore. But instead, this sample shows a broad peak in the range of 245° C.-250° C., whose area clearly exceeds that of the physical mixture. This peak indicates the decomposition of the supramolecular complex agglomerate. These agglomerates lead to poor dissolution behavior in that a “spring parachute effect” occurs due to the formation of supramolecular agglomerates, whereby the complex precipitates out of the solution after dissolution.

### 4. Saturation Solubility of Cyclodextrin Complexes in Distilled Water (HPLC)

The final most important point to compare the manufacturing methods is the solubility in distilled water. This is because the solubility of the complex has a direct influence on the bioavailability, since only dissolved complexes/active substances can pass through the epithelial cells of the GI tract. In addition, the samples were analyzed for related substances to detect possible breakdown of the active substance during the preparation process.

### Method:

### Reference Measurement (Taxifolin)

10 mg of taxifolin (Lavitol® 98.9% purity) were added to a vial containing 5 ml distilled water to produce a saturated solution and was shaken for 60 min. The solution was then transferred to a vial by syringe with HPLC filter (0.22 μm) and then measured in undiluted state (HPLC DAD-254 nm).

### Sample Measurement

500 mg of the sample were added to a vial containing 6 ml distilled water to produce a saturated solution and was shaken for 60 min. In the following, the solution was transferred to a vial using a syringe with HPLC filter (0.22 μm), diluted 10:1 with distilled water to avoid supersaturation and then measured (HPLC DAD-254 nm). Based on the peak area taking into account the dilution, the taxifolin concentration was calculated in mg/ml.

### Results:

| | Solubility | Rel. |
|---|---|---|
| | taxifolin | Substances |
| Name | in mg/ml | Peak |
| Taxifolin reference | 0.7496 | 3430343 |
| Phys. Mix 1:1 β-CD | 22.3834 | — |
| Phys. Mix 1:1 γ-CD | 5.1944 | |
| SD β | 24.8682 | — |
| FD β | 23.7192 | — |
| FD γ | 5.0152 | |

The saturation solubility of the flavonoid was increased by inclusion complexes with β-CD and also, to a lesser extent, with γ-CD. This effect is particularly pronounced in the spray-dried SD β formulation. However, the saturation solubilities of the γ-CD complexes are significantly lower than those of the β-CD complexes.

The physical 1:1 mixtures also provided very good results, which can be attributed to the complex formation in solution. The physical mixture actually represents the maximum possible upper limit for solubility enhancement, since here the complex can form under maximum saturation, i.e., optimal conditions.

Nevertheless, the taxifolin concentration of the SD β formulation exceeds this value. This is probably due to supersaturation of the solution due to the small particle size and thus large surface area of the material.

5. Agglomeration in Complexes with γ-CD

An important point to consider, especially for γ-cyclodextrins, is possible agglomeration of the complexes. This problem has an enormous influence on the solubility and dissolution behavior of the product. In this case, the complexes arrange themselves into supramolecular complexes in a solid crystal structure. This massively reduces the surface area and also the hydration of the individual complexes. Consequently, even if there is high solubility of the complexes in theory, a turbid, characteristically opalescent suspension is formed.

In order to be able to demonstrate the solubility restriction by agglomeration properly, experiments were carried out with chaotropic substances. These substances prevent the formation of hydrogen bonds, which stabilize the complexes in the highly ordered structure. At the same time, the highly ordered structure of the solvent water is broken, and thereby hydrophobic effects are reduced.

Specifically, an opalescent suspension of a γ-CD complex was prepared (250 mg of γ-CD complex powder in 20 ml distilled water) again, and then 10 g urea was added. The suspension completely cleared after only 10 min of stirring at 600 rpm without increasing the temperature. By breaking up the aggregates, the solubility could be considerably increased.

These agglomerates do not occur in β-CD, so only β-CD is suitable to ensure optimal resorption of the flavonoid and thiamine. This is due to the instant-release behavior of this formulation, whereby negative interactions of the taxifolin with intestinal thiamine transporters can be reduced.

### 6. Ternary Complexes β-Cyclodextrin

In order to examine which water-soluble polymers are particularly suitable for improving the stability and dissolution capacity of flavonoid-cyclodextrin complexes, a screening was carried out. For this purpose, first, a supersaturated taxifolin/β-CD complex solution was prepared by adding an excess of equimolar taxifolin/β-CD complex and subsequent heating to 35° C. and filtering off. Subsequently, various water-soluble polymers were added (0.25% w/v) and choline bitartrate as well as L-carnitine tartrate were added (taxifolin:choline/carnitine cations ratio 1:0.85) to examine the influence of polymers/alkylammonium cations on complex formation and solubility, respectively. The solution was allowed to stand for 96 h and then recrystallization was compared with the reference solution.

| Polymer used | Recrystallization after | |
|---|---|---|
| (0.25% w/v) | 96 h | Remark |
| No polymer (reference) | Distinct | Reference |
| PVP K30 | Very strongly distinct | Deterioration |
| PVP/VA | Very strongly distinct | Deterioration |
| HPMC | Very distinct | Deterioration |
| MC | Distinct | No change |
| Carbomer | Distinct | No change |
| Poloxamer 188 | Not present | Improvement |
| PEG 6000 | Not present | Improvement |
| PVA | Merely present | Improvement |
| PEG/PVA (Kollicoat ® IR) | Not present | Improvement |
| Xanthan | Distinct | No change |
| Gellan | Distinct | No change |
| Eudragit E100 | Very distinct | Solution in 0.1N |
| | | HCl, Deterioration |
| Chitosan | Distinct | Solution in 0.1N |
| | | HCl, no change |
| Pectin | Distinct | No change |
| Na-CMC | Very distinct | Deterioration |
| Alginic acid | Distinct | No change |
| Collagen Hydrolysat | Distinct | No change |
| Maltodextrin | Distinct | No change |
| Choline bitartrate | Not present | Improvement |
| L-Carnitine tartrate | Distinct | No change |

The results clearly show that polymers with prominent H-bridge acceptors (PVP, PVP/VA, Eudragit E100 and cellulose derivatives) lead to breakdown due to a too strong interaction with the drug. The polymer-drug complex precipitates and Ks decreases. In addition, no interaction could be detected for typical biopolymers, either with regard to the active substance or with the cyclodextrin, and, therefore, the dissolution behavior of the active substance is not changed.

In contrast, PEG 6000, Kollicoat IR and Poloxamer 188 are of particular interest. These polymers are composed of ethylene oxide blocks and show very promising properties. The interaction with the hydroxy groups of the flavonoid are not so strong that precipitation occurs. At the same time, the polymers also interact with the hydroxy groups of the cyclodextrin. This increases the complex stability. The same can be seen with polyvinyl alcohol (PVA). However, the interaction of the hydroxyl groups of the polymer with the flavonoid and the cyclodextrin is less pronounced than with the ethylene oxide polymers.

This showed that the use of water-soluble polymers can increase the complex stability and improve the dissolution behavior.

Furthermore, a significant improvement was observed when choline bitartrate was added, however, this was not the case for the structurally related L-carnitine.

Thereby, it could be demonstrated that not every alkylammonium cation, but only choline cations are suitable for this purpose. This can be explained by the structure-breaking influence of the alkylammonium group on the hydrogen bonds of the solvent and the associated single-salt effect. As regards carnitine, on the other hand, the hydroxyl group as well as the carboxyl group act as structure-forming elements which can form H-bridges and counteract the effect of the alkylammonium group. It was found that in choline compounds, on the other hand, the structure-breaking component predominates and leads to an improvement in solubility and physicochemical properties, respectively, especially in taxifolin/β-CD formulations as well as in solid dispersions of taxifolin/basic polymethacrylate.

In order to achieve this positive effect, it is already sufficient to physically mix or combine the water-soluble polymer/choline compound and the final flavonoid/CD complex in an oral dosage form, since a ternary complex is formed after dissolution in solution and the positive effect of the choline cation unfolds, respectively. However, integration of the polymer can also occur before or during complex formation. For example, small amounts of the polymer can be added to the complex solution before spray or freeze drying.

7. Preparation of Solid Dispersions with Eudragit® E

### Common Solvent Evaporation 2:1 (CSE 2:1)

2000 mg of Eudragit® E100 was weighed out and dissolved in 30 ml ethanol. Subsequently, 1000 mg of taxifolin were weighed out and dissolved in 15 ml ethanol. Hereafter, both solutions were mixed and stirred at 600 rpm and at room temperature for 30 min. At last, the clear, light amber solution was dried in a dry place protected from light. After powdering, the solid dispersion was stored airtight and protected from light.

### XRD Analysis

The XRD method is considered the method of choice for detecting the complete, amorphous embedding of an active substance in the polymer matrix. For this purpose, the crystallinity of the sample is determined, which provides conclusions about the arrangement of the molecules of the active substance. Since contrary to the active substance, the polymer matrix is amorphous, crystalline peaks indicate incomplete embedding. If, however, the sample is amorphous, a solid solution is present.

In addition, amorphous samples usually show significantly better dissolution behavior than crystalline samples, which is why an increase in bioavailability is possible with an amorphous sample.

Result: It can be taken from the diffraction diagrams that both taxifolin and the physical mixture of taxifolin/Eudragit® E100 are crystalline. As expected, the polymer is amorphous. The physical mixture also shows superimposed X-ray diffraction patterns of taxifolin and Eudragit® E100. Furthermore, all three formulations are amorphous and do not differ from the reference polymer.

Discussion: The results of the XRD analyses indicate that a solid dispersion is present at CSE 2:1, with the flavonoid taxifolin being fully embedded in the polymer matrix.

### FITR Analyses

FT-IR spectroscopy is applied in order to analyze the molecular interactions between the functional groups of the flavonoid and the basic polymethacrylate.

Initially, the peak is broadened at 3435 cm⁻¹, which is due to the presence of a protonated ammonium group as the R—N+-H stretching vibration absorbs in exactly this region, thus broadening the band. This indicates that the tertiary amino group of the polymer is present in protonated form. Also, as regards the peaks at 2770 cm⁻¹ and 2820 cm⁻¹ a significant loss of intensity or even complete disappearance occurs, which implies that the tertiary amino group of the polymer is involved in ionic interactions with the flavonoid.

There exist strong ionic interactions between the tertiary amino groups of the polymer and the phenolic hydroxyl groups of the flavonoid, whereby the tertiary amino groups are protonated to cationic ammonium groups and the hydroxyl groups of the flavonoid are deprotonated to resonance-stabilized phenolate ions.

8. Solubility of the Solid Dispersion with Eudragit E

The last most important point in order to compare the preparation methods is the solubility in simulated gastric juice. The solubility of the complex has a direct influence on the bioavailability, because only dissolved active substances can pass through the epithelial cells of the GI tract.

### Reference Measurement (Taxifolin)

10 mg of taxifolin (Lavitol® 98.9% purity) was added to a vial containing 5 ml 0.1N HCl to produce a saturated solution and was shaken for 60 min. Subsequently, the solution was transferred to a vial by means of a syringe with HPLC filter (0.22 μm) and then measured.

### Sample Measurement

A saturated solution of the sample was prepared in 0.1 molar HCL solution at room temperature. In the following, the solution was transferred to a vial by means of a syringe with an HPLC filter (0.22 μm), diluted accordingly, and the taxifolin concentration of the solution was determined by HPLC.

| | Initial | | Solubility |
|---|---|---|---|
| | weight | Dilution | of taxifolin |
| Name | mg | factor | in mg/ml |
| Taxifolin reference | 10.34 | — | 0.6927 |
| CSE 2:1 | 1331.63 | 66.6 | 15.00 |

Discussion: By formulating a solid dispersion with basic polymethacrylates the saturation solubility of taxifolin can be increased. This is particularly due to the fact that the flavonoid is embedded in amorphous form in the polymer in all three formulations, which is confirmed by both FT-IR and XRD analyses.

9. Dissolution Behavior of Formulations with Cyclodextrin or Basic Polymethacrylate

In order to examine the dissolution behavior of the final formulations, dissolution studies of the cyclodextrin and eudragitol formulations against pure taxifolin were carried out. Here, the instant-release formulations are expected to significantly improve the dissolution behavior of the flavonoid, as the pure taxifolin dissolves quite slowly due to its stable crystalline structure and low water solubility.

Due to the solid dispersion with Eudragit® E the crystalline structure is dissolved (see XRPD analyses) and thus water solubility is increased. In the case of the CD complexes, the crystalline structure is also dissolved by encapsulating each individual taxifolin molecule, and at the same time the water solubility and wettability are increased by the CD acting as a “Trojan horse”. Both should lead to an improvement in dissolution behavior.

The instant-release formulation is considered optimal when 85% of the drug has dissolved within the first 15 min. Since gastric emptying when fasting is a first order reaction (50% emptying in 10-20 min), 85% dissolution within the first 15 min, it can be assumed that the formulation behaves like a solution and, thus, behaves optimally. Thereby, optimal absorption behavior of thiamine and taxifolin can be ensured when administered at the same time.

Method: In order to determine the dissolution behavior, the usual pharmacopoeial procedure was chosen.

USP Apparatus II (paddle); 100 rpm; medium: 500 ml 0.1N HCl; 2 vessels per sample (N=2); 7 sampling points: 0 min, 5 min, 10 min, 15 min, 20 min, 30 min, 60 min; weight: formulation as powder corresponding to 100 mg of taxifolin; detection by HPLC.

The following formulations were tested:
taxifolin (Ametis Lavitol®, 98.8% purity)
Eudragit® E solid dispersion formulation
β-cyclodextrin formulation

Here, the pure taxifolin represents the reference value.

### Results:

Taxifolin release (reference)

| | | | Initial | | |
|---|---|---|---|---|---|
| Sampling | | | weight | | Mean |
| Time | | Vessel | mg | Release | Release |
| 5 | min | 1 | 106.52 | 28.2% | 29% |
| | | 2 | 108.20 | 29.2% | |
| 10 | min | 1 | 106.52 | 46.4% | 46% |
| | | 2 | 108.20 | 44.8% | |
| 15 | min | 1 | 106.52 | 61.1% | 60% |
| | | 2 | 108.20 | 58.7% | |
| 20 | min | 1 | 106.52 | 70.4% | 69% |
| | | 2 | 108.20 | 67.5% | |
| 30 | min | 1 | 106.52 | 80.8% | 79% |
| | | 2 | 108.20 | 77.4% | |
| 60 | min | 1 | 106.52 | 90.8% | 92% |
| | | 2 | 108.20 | 93.1% | |

| | | | | |
|---|---|---|---|---|
| | | Initial | | |
| Sampling | | weight | | Mean |
| Time | Vial | mg | Release | Release |

Release taxifolin/β-CD complex

| | | | | | |
|---|---|---|---|---|---|
| 5 | min | 1 | 586.93 | 100.1% | 100% |
| | | 2 | 587.81 | 100.5% | |
| 10 | min | 1 | 586.93 | 100.6% | 100% |
| | | 2 | 587.81 | 100.2% | |
| 15 | min | 1 | 586.93 | 103.2% | 103% |
| | | 2 | 587.81 | 103.1% | |
| 20 | min | 1 | 586.93 | 100.1% | 100% |
| | | 2 | 587.81 | 100.5% | |
| 30 | min | 1 | 586.93 | 100.5% | 101% |
| | | 2 | 587.81 | 101.1% | |
| 60 | min | 1 | 586.93 | 100.3% | 101% |
| | | 2 | 587.81 | 101.3% | |

Release Eudragit ® E solid dispersion

| | | | | | |
|---|---|---|---|---|---|
| 5 | min | 1 | 301.21 | 82.2% | 82.2% |
| | | 2 | 303.27 | (150.0%) | |
| 10 | min | 1 | 301.21 | 84.8% | 85% |
| | | 2 | 303.27 | 84.5% | |
| 15 | min | 1 | 301.21 | 86.6% | 86% |
| | | 2 | 303.27 | 86.2% | |
| 20 | min | 1 | 301.21 | 85.5% | 85% |
| | | 2 | 303.27 | 84.4% | |
| 30 | min | 1 | 301.21 | 85.3% | 85% |
| | | 2 | 303.27 | 84.9% | |
| 60 | min | 1 | 301.21 | 85.0% | 85% |
| | | 2 | 303.27 | 84.8% | |

Note:
At time of sampling 5 min, vial 2, a particle was drawn through the filter, which dissolved before the measurement. Therefore, this measurement point was therefore not taken into account.

Discussion: Taxifolin in its free form shows a typical dissolution behavior with continuous release. The results are shown in FIG. 2. However, the release after 15 min is only 60% and thus does not meet the requirement of an instant-release formulation (min. 85% after 15 min). This means that reduced thiamine resorption is to be expected. Both the solid dispersion in Eudragit® E and the β-cyclodextrin formulation meet the requirements and are, therefore, considered optimal instant-release formulations.

β-CD releases the flavonoid very quickly and already achieves 100% release at the first measuring point. Furthermore, there is no recrystallization in the sense of a “spring parachute effect” as occurs in γ-CD complexes, but the release is constantly 100%.

The Eudragit® E formulation also achieves a very rapid release of the flavonoid, with 82.2% of the flavonoid already in solution at the first measuring point. Here, too, there is no recrystallization and no precipitation of the taxifolin from the solution, but the release of the taxifolin is limited to a maximum of 85%.

Both formulations thus fulfil the requirements for optimal instant-release formulations, which allow the formulation of a taxifolin/thiamine combination.

In addition, both formulations allow good storage stability by unwanted redox reactions between the taxifolin and the thiamine during the storage period being able to be avoided. This is due to the inclusion of the catechol group in the β-CD formulation, while ionic interactions between the hydroxyl groups of the catechol group and the aminoalkyl moiety of the polymer are decisive in the solid dispersion in basic polymethacrylate.

### 10. Stability Experiments Thiamine

Stability experiments were carried out in order to investigate the interactions between thiamine and taxifolin and the influence of galenic formulations in more detail. Contrary to the breakdown of taxifolin, the breakdown of thiamine is not accompanied by a color change and is therefore more difficult to detect. However, the possible breakdown products, in particular thiamine disulfide as well as under certain circumstance also thiochrome, have completely different physicochemical properties, which can be exploited by thin-layer chromatography.

Method: First, four mixtures were prepared in a mortar consisting of I) 1000 mg taxifolin and 127 mg thiamine HCl II) 5266 mg taxifolin/γ-CD complex (FD-γ) and 127 mg thiamine HCl III) 4730 mg taxifolin/β-CD complex (FD β) and 127 mg thiamine HCl and IV) 3030 mg taxifolin/Eudragit® E CSE 2: 1 and 127 mg thiamine HCl, wherein each formulation contained 1000 mg taxifolin and the thiamine HCl corresponded to 100 mg thiamine (taxifolin:thiamine ratio 10:1).

The mixtures were placed in glass petri dishes and stored open in a climate-controlled cabinet at 40° C. and 75% humidity for 3 months (Accelerated Stability Test).

In the following, the samples were divided in half and the amount corresponding to 50 mg of thiamine was weighed out in each case (564 mg of taxifolin/thiamine, 2697 mg of FD-γ/thiamine, 2429 mg FD β/Thiamine and 1579 mg of Eudragit® E CSE 2:1/thiamine). Subsequently, each sample was extracted with 50 ml of solvent having a temperature of 45° C. (ethanol for the taxifolin-pur, FD-γ and FD β mixture and petroleum ether for the Eudragit® E CSE 2:1 mixture) in order to dissolve the thiamine breakdown products, and then filtered. The final solutions contained the equivalent amount of breakdown product of 50 mg of thiamine/50 ml solvent.

Besides, a reference solution was prepared containing the equivalent concentration of thiamine disulfide (53 mg of thiamine disulfide hydrate in 50 ml ethanol and petroleum ether, respectively).

In the following, silica gel DC plates were loaded with 5 μl per sample each and placed in a DC chamber along with a running medium consisting of ethanol:acetone:acetonitrile 4:2:1. The plates were dried thereafter and sprayed with Dragendorff reagent. The Dragendorff reagent was chosen because it specifically stains basic tertiary amines due to the potassium tetraiodobismuthate complex it contains. This allows selective staining of thiamine as well as its breakdown products thiamine disulfide and thiochrome.

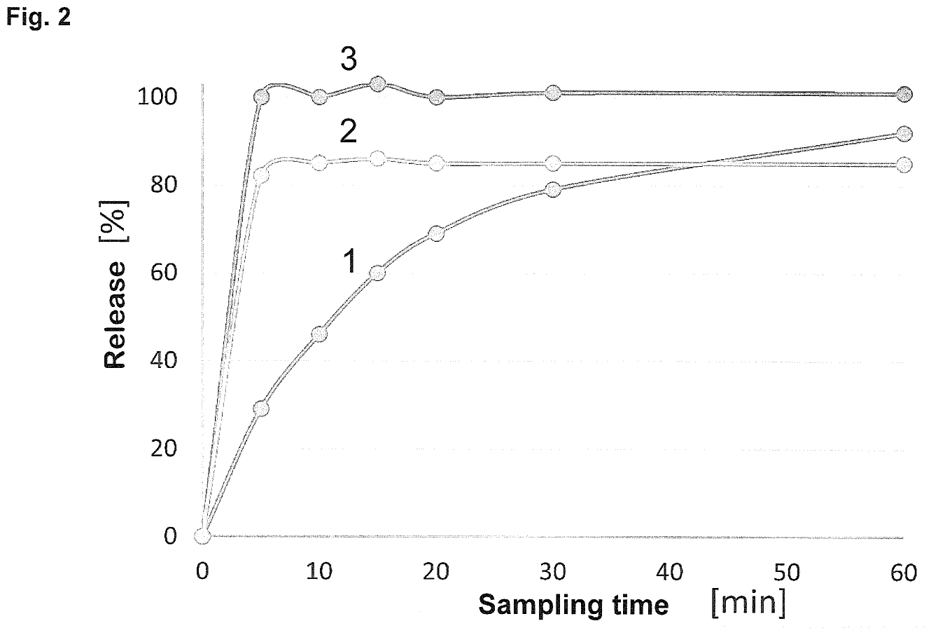

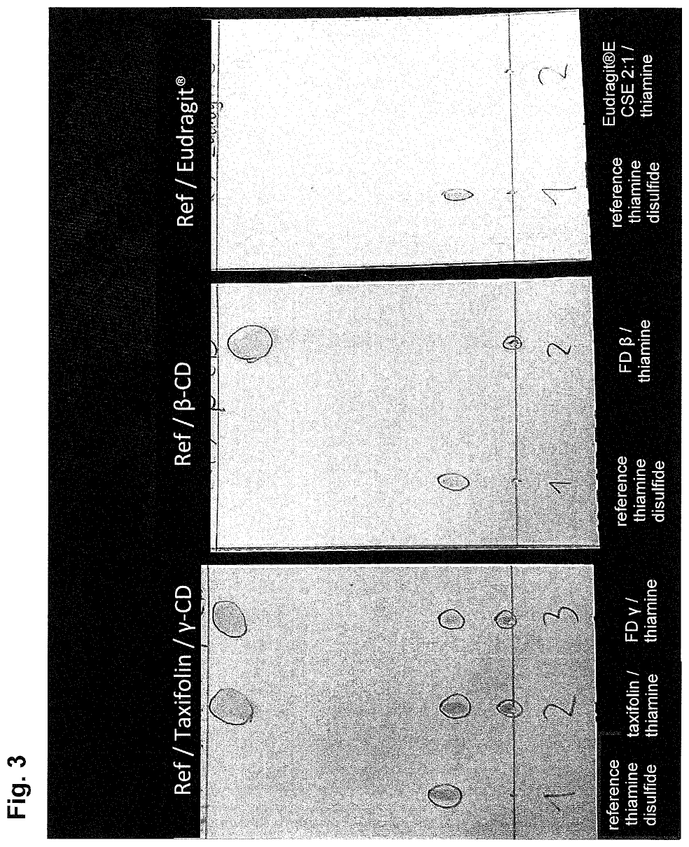

Results: Thin layer chromatography resulted in a clean separation of the substances (FIG. 3), in particular a clear, semi-quantitative detection of thiamine disulfide. Taxifolin was entrained with the running medium and is visible near the running centerline due to oxidation in air, but thiamine HCl remained at the starting line due to its hydrophilicity instead. Thiamine disulfide was cleanly separated and had an Rf value in the optimum range of 0.22 to 0.27.

Thiamine disulfide could be detected in the taxifolin/thiamine and FD-γ/thiamine mixtures, whereby less breakdown was visible in the FD-γ than in the pure taxifolin/thiamine sample. In contrast, no thiamine disulfide or other breakdown product was present in the FD β sample or in the Eudragit® E CSE 2:1 sample. Thiochrome could not be detected in any sample under UV light.

Discussion: The taxifolin formulations with β-CD and basic polymethacrylate were the only formulations which were able to inhibit the breakdown of thiamine to thiamine disulfide. This is due to the encapsulation of the catechol group by β-CD or the ionic interactions between taxifolin and the basic polymethacrylate. The sample containing Eudragit® E had to be extracted with petroleum ether, as otherwise the polymer would also have dissolved and been stained by the Dragendorff reagent. By this extraction no polymer, thiamine HCl or taxifolin became visible in the Eudragit® E sample, as these are too polar for the extracting agent petroleum ether, in contrast to the lipophilic thiamine disulfide, which could be extracted in the reference solution. In addition, the thiamine HCl in the taxifolin/thiamine sample and in the FD-γ/thiamine sample runs slightly further than in the FD β sample. This is probably due to interactions between thiamine and the β-CD, which increase the hydrophilicity of the vitamin.

### 11. Stability Examination Taxifolin

In order to investigate the stability of the flavonoid taxifolin and the influence of thiamine and β-CD, experiments were also carried out in this regard. Since taxifolin forms red-brown oligomers upon breakdown, the detection is quite straightforward to carry out.

Method: Three aqueous solutions were prepared in beakers containing I) 100 mg taxifolin in 150 ml distilled water II) 100 mg of Taxifolin+13 mg of thiamine HCl in 150 ml distilled water and III) 100 mg taxifolin+13 mg of thiamine HCl+373 mg of β-CD in distilled water. Samples were stored open and protected from light at room temperature and the color of the solution was checked every 24 h.

Results: The results are summarized in the following table.

| | Time of | Color of |
|---|---|---|
| Sample | color change | solution |
| Taxifolin (Ref.) | 48 h | Red-brown |
| Taxifolin/thiamine | 48 h | Yellowish, brown |
| | | staining after 72 h |
| Taxifolin/thiamine/β-CD | 96 h | Yellow-brown |

A delay in taxifolin oxidation by addition of thiamine or β-CD can be seen, with oxidation decreasing in the order taxifolin (ref.)>taxifolin/thiamine>taxifolin/thiamine/β-CD.

Discussion: Addition of thiamine can delay the breakdown of taxifolin, with thiamine disulfide and various breakdown products and/or adducts being formed in the process, causing the yellow coloration of the solution. This also confirms the beneficial combination in vivo, wherein thiamine can reduce oxidized taxifolin, and thus prolongs the effect. The addition of R-CD now delays taxifolin oxidation in the first step, which results in in delayed oxidation of thiamine.

12. Oral Dosage Form with β-Cyclodextrin, Thiamine and Choline

Dosage corresponds to 1 tablet, ingredients per tablet, oblong shape:

| Ingredient | Dosage/Tablet (mg) |
|---|---|
| Taxifolin/β-CD complex spray-dried | 500 |
| (containing 20% taxifolin from larch | |
| extract) | |
| thiamine HCl | 13 |
| choline bitartrate | 207 |
| Microcrystalline cellulose | 118 |
| Polyethylene glycol 6000 | 25 |
| Magnesium stearate | 6 |

The parameters of the finished tablet are as listed below:

| Parameter | Result | |
|---|---|---|
| Height | 6.05 | mm |
| Breadth | 8.5 | mm |
| Depth | 20 | mm |
| Mass | 869 | mg |
| Pressure force for production | 12 | kN |
| Tablet hardness (longitudinal) (N = 10) | >280N | |
| Disintegration time (N = 6) | 16.5 | min. |

| | |
|---|---|
| Abrasion/Friability (N = 10) | 0.023% |

The results illustrate that the taxifolin formulation with R-CD, choline and thiamine can also be easily produced on a large scale, whereby the parameters are in the optimal range. In addition, the thiamine can now be in microencapsulated form, for example.

13. Formulation with Basic Polymethacrylate and Thiamine

Dose corresponds to 1 hard capsule, ingredients per hard capsule size 0 (gelatin):

200 mg of basic methacrylate copolymer (Eudraguard Protect®, Evonik Nutrition & Care GmbH), 100 mg of taxifolin-rich extract from Larix gmelinii (Lavitol® from Ametis JSC, taxifolin content 90.5%), 20 mg of silicon dioxide, 13 mg of thiamine hydrochloride (Food Grade, BASF).

The formulation with basic polymethacrylate is also easy to implement and can be produced on a large scale.

14. Formulation with β-Cyclodextrin+Thiamine Microencapsulated

Dose corresponds to 1 tablet, ingredients per tablet, oblong shape 21 mm×9 mm:

740 mg of β-cyclodextrin (Food Grade, CycloLab R&D Ltd.), 200 mg of taxifolin-rich extract of Larix gmelinii (Lavitol®, Ametis JSC, taxifolin content 90.5%), 35 mg of silica, 30 mg of thiamine microencapsulated (33.3% thiamine HCl+66.6% carnauba wax white), 20 mg of polyethylene glycol 6000.

1. A formulation for oral administration comprising.
(i) taxifolin or a pharmaceutically acceptable salt, derivative or prodrug thereof,
(ii) thiamine or a pharmaceutically acceptable salt, derivative or prodrug thereof, and
(iii) at least one excipient selected from a) β-cyclodextrin and derivatives thereof, and b) a basic (co)polymer of methacrylic acid and/or methacrylate,
wherein taxifolin is present (a) as a complex with the β-cyclodextrin or derivative thereof, or (b) as a solid dispersion with the basic (co)polymer of methacrylic acid and/or methacrylate.

2. The formulation according to claim 1,
wherein taxifolin is present as a complex with β-cyclodextrin or a derivative thereof, preferably in a molar ratio of about 1:1, and wherein derivatives are selected from substituted ß-cyclodextrins that are substituted on one or more hydroxyl groups, in particular on the C6 carbon atom of one or more glucose units, preferably with —O—C₁₋₁₈ alkyl or —O—C₁₋₁₈ hydroxyalkyl groups.

3. The formulation according to claim 1,
wherein taxifolin is present as a solid dispersion with the basic (co)polymer of methacrylic acid and/or methacrylate, preferably in a weight ratio of taxifolin:basic (co)polymer of methacrylic acid and/or methacrylate in the range of 1:1 to 1:3, wherein the basic (co)polymer of methacrylic acid and/or methacrylate is preferably selected from Eudragit® E and Eudraguard Protect®.

4. The formulation according to claim 1, wherein thiamine is present as mononitrate or hydrochloride, preferably in microencapsulated form.

5. The formulation according to claim 1, wherein taxifolin is present in the form of an extract of larch wood, preferably an extract of Dahurian larch (Larix gmelinii), wherein the extract may optionally comprise one or more additional flavonoids, preferably aromadendrin and/or eriodictyol.

6. The formulation according to claim 5,
wherein the taxifolin content in the extract is at least 88%, preferably 90-97%, more preferably 90-93%.

7. The formulation according to claim 1, wherein taxifolin is present in an amount of 50-500 mg, more preferably 50-150 mg, and/or thiamine is present in an amount of 0.1-250 mg, preferably 1-100 mg, particularly preferably 5-50 mg.

8. The formulation according to claim 1, wherein the taxifolin:thiamine ratio is in the range of 700:1 to 1:1, preferably in the range of 100:1 to 3:1, more preferably in the range of 20:1 to 5:1, and most preferably in the range of 10:1.

9. The formulation according to claim 1, further comprising a water soluble polymer, preferably selected from polyethylene glycol, polyvinyl alcohol, poloxamer and mixtures thereof.

10. The formulation according to claim 1,
further comprising one or more pharmacologically acceptable excipients and/or carriers, and/or
one or more further ingredients, preferably selected from choline, vitamins, in particular B vitamins, vitaminoids, minerals, trace elements, amino acids and pharmaceutically acceptable salts, derivatives and prodrugs thereof.

11. The formulation according to claim 1, wherein the formulation is present in the form of powder, granules, capsule, tablet, chewable tablet, effervescent tablet, coated tablet, sachet or solution/suspension, wherein the formulation may consist of one or more dosage units, wherein preferably at least one dosage unit is in the form of compressed material.

12. The formulation according to claim 1, for the use as a medicament.

13. The formulation according to claim 1, for the use in the prevention or treatment of alcohol intoxication, consequential conditions and secondary diseases associated with alcohol consumption, or alcoholism.

14. The formulation for use according to claim 13,
wherein consequential conditions associated with alcohol consumption comprise hangovers.

15. The formulation for use according to claim 1, wherein consequential conditions and diseases associated with alcohol consumption comprise damage due to alcohol intoxication, in particular neurological damage as well as liver damage.

16. The formulation for use according to claim 13,
wherein the treatment of alcoholism comprises alcohol dishabituation and/or alcohol withdrawal.
